(19) United States
(12) Patent Application Publication
(10) Pub. No.: US 2021/0186648 A1
(43) Pub. Date: Jun. 24, 2021

(54) SURGICAL SHAPE SENSING FIBER OPTIC APPARATUS AND METHOD THEREOF

(71) Applicants: Yan XIA, West Lafayette, IN (US); Lu LAN, West Lafayette, IN (US); VIBRONIX, INC., West Lafayette, IN (US)

(72) Inventors: Yan XIA, West Lafayette, IN (US); Lu LAN, West Lafayette, IN (US)

(21) Appl. No.: 16/754,800

(22) PCT Filed: Oct. 10, 2018
(86) PCT No.: PCT/US18/55229

§ 371 Date: Apr. 9, 2020

(51) Int. Cl. A61B 90/00; G06T 7/20

(52) U.S. Cl. CPC A61B 90/37; A61B 90/361; G06T 7/20; A61B 2090/373; G06T 2207/30204; A61B 2090/365; A61B 2090/367; G06T 2207/10012; A61B 2090/3614

(57) ABSTRACT

A shape sensing apparatus for tissue and surgical procedures comprising a processing means and a tunable light source. At least one shape sensing fiber can be used with the shape sensing fiber having a plurality of individual sensing fiber cores having a fiber Bragg grating distributed within the fiber. An optical switch configured to sequentially switch between a multiplex of individual fibers inside the shape sensing fiber for signal detection can be included and a detector can be used to detect the fiber signals. An augmented reality system that receives the tracking data from the shape sensing apparatus, and superimposes visual guidance on its display for precise and intuitive surgical guidance.

### CROSS-REFERENCE TO RELATED APPLICATION

This patent application claims priority to U.S. Provisional Application 62/570,217 filed Oct. 10, 2017, the disclosure of which is considered part of the disclosure of this application and is hereby incorporated by reference in its entirety.

### FIELD OF THE INVENTION

This invention relates generally to shape sensing fiber optics, more particularly to sensing position and orientation at a plurality of points of surgical guidance of a surgical instrument during an operation.

### BACKGROUND

Surgical procedures can range invasiveness and many surgical procedures being carried out today that were once greatly invasive, are now becoming less and less invasive. The miniaturization of medical devices has enabled the development of new approaches for the diagnosis and treatment of human disease. Endoscopic devices is a profound example to allow for minimally invasive surgical procedures with very small incisions to be made to carry out the procedure. Also, various medical devices/implants are being developed or used to perform procedures which are not easily accessible through conventional surgical instrument. For example, smart pills are being used to image the gastrointestinal tract. While endoscopic or microscale devices are capable of sensing their environment and performing interventions, such as biopsies, it is important to precisely determine the location and geometry of the endoscopic devices inside the human body.

Some have tried to measure shape changes by using foil strain gauges. These sensors, while sufficient for making local bend measurements, are impractical for use with sufficient spatial resolution to reconstruct shape or relative position over all but the smallest of distances. Others have used fiber optic micro-bend sensors to measure shape. This approach relies on losses in the optical fiber which cannot be controlled in a real-world application. Clements (U.S. Pat. No. 6,888,623 B2) describes a fiber optic sensor for precision 3-D position measurement. The central system component of the invention is a flexible “smart cable” which enables accurate measurement of local curvature and torsion along its length. These quantities are used to infer the position and attitude of one end of the cable relative to the other.

Similarly, Chen et al. (U.S. Pat. No. 6,256,090 B1) describe a method and apparatus for determining the shape of a flexible body. The device uses Bragg grating sensor technology and time, spatial, and wavelength division multiplexing, to produce a plurality of strain measurements along one fiber path. Using a plurality of fibers, shape determination of the body and the tow cable can be made with minimal ambiguity. The use of wavelength division multiplexing has its limitations in that the ability to have precision with respect to determining the shape and/or position of an object is limited. Wavelength division multiplexing can only be used with sensor arrays that have less than one hundred sensors and, therefore, is insufficient for the application of determining shape and or position of an object with any precision.

However, the localization of cancerous or other target tissue for excision can be difficult and result in more tissue being removed than necessary. Current methods are incapable of providing quantitative location of the implanted device or real-time visual feedback of that location. This creates problems on a large re-excision rate, and a prolonged surgical time. These factors subsequently result in higher surgical cost/waste, high risk of complication, and physical pain and emotional distress for the patients. Thus, there is an unmet need for an effective surgical imaging apparatus that can provide real-time accurate visualization of the excision area and generate a visual guidance for surgical instruments within a patient during surgical procedures.

### BRIEF SUMMARY OF THE INVENTION

In one aspect, this disclosure is related to a shape sensing apparatus for tissue and surgical procedures comprising a processing means and a tunable light source. At least one shape sensing fiber can be used with the shape sensing fiber having a plurality of individual sensing fiber cores having a fiber Bragg grating distributed within the fiber. An optical switch configured to sequentially switch between a multiplex of individual fibers inside the shape sensing fiber for signal detection can be included and a detector can be used to detect the fiber signals. Similarly, multiple modules can be used in parallel to simultaneously acquire signal from multiple fiber cores or fibers inside the shape sensing fiber. A data acquisition module can digitize the detected signals and communicate the digitized signals to the processing means. The processing means can then reconstruct a 3D shape based on the signals. A first group of one or more tracking markers can be coupled to a proximal terminator of the shape sensing fiber. In one embodiment, the markers can be configured to actively emit a signal, such as IR light or passively reflect light from an IR light source. Similarly, the markers can use electromagnetic signals. A spatial tracking means, such as a stereo camera or electromagnetic (EM) tracking means, can be configured to detect and track the first group of one or more markers within a predetermined area can be used by the apparatus. The spatial tracking means, such as a stereo camera, can include a second light source to locate the proximal terminator of the shape sensing fiber having a passive IR marker. Similarly, the second light source 180 can be used to locate other IR markers of the system, specifically passive IR markers that need to be illuminated by the second light source. The secondary light source can be optional in some embodiments of the present invention. Embodiments utilizing an active IR marker or an EM marker may not require the secondary light source as they can produce their own signal to be detected by the spatial tracking means.

Various embodiments of the system of the present disclosure can use different markers, including passive IR markers that reflect a light signal, active IR markers that produce their own light signal, and EM markers that produce an electromagnetic signal. Each of the different signals can be detected using a spatial tracking means. In some exemplary embodiments, the signal produced by the markers can be any suitable signal, such as reflected IR light, IR light, or an EM signal.

The processing means may then determine the pose and position of the proximal terminator relative to the spatial tracking means and combine this position data with the reconstructed 3D data to determine the 3D shape of the sensing fiber relative to the spatial tracking means. The apparatus of the present disclosure can then use a camera to obtain real-time image/video of the tissue being operated on by a physician or medical technician. A display may be provided to render the shape of the sensing fiber and superimpose the shape over the view of the tissue being operated in real-time. A second group of one or more markers may be mounted to a surgical instrument. The second group of one or more markers may be tracked by the spatial tracking means, wherein the obtained tracking data from the spatial tracking means is communicated to the processing means and the spatial relation of the surgical equipment relative to the 3D shape sensing fiber inserted in the tissue is obtained and may be displayed on a display, such as a tablet or head mounted display (HMD) viewed by the physician or medical technician. Tracking means can include any suitable means, such as infrared and EM tracking. In one embodiment, EM markers can replace the second group of infrared markers to be mounted on surgical instrument for tracking.

The memory communicatively coupled to the processing means can include a shape construction module to reconstruct three dimensional shapes resulting in 3D shape data of the fiber from determined locations, a spatial geometry module configured to determine the location of the shape sensing fiber relative to the spatial tracking means; an optical tracking module configured to collect the optical images of the first group of markers and the second group of markers to calculate the spatial pose and position data of the markers with respect to the spatial tracking means, and a data streaming module configured to transmit the spatial pose and position data, the 3D shape data to be displayed on the display.

In another aspect, this disclosure is related to a method of providing an augmented reality surgical system comprising at least one shape sensing fiber and 3D visualization system as disclosed. A shape sensing fiber having Bragg grating can then be inserted into a target tissue or pre-determined area of a patient. In addition, the shape sensing fiber having Bragg grating can be mounted or inserted in a target surgical instrument to be tracked. A light source can be used and ran through the shape sensing fiber, wherein the fiber has fiber Bragg gratings at one or more locations of the fiber within the patient. The reflectivity at one or more wavelengths of the one or more fiber Bragg gratings at one or more locations of the fiber with the patient can then be measured. The strains on the fiber Bragg gratings at different locations can be determined. A three dimensional shape of the fiber from the determined locations can then be generated. A display can be used to present the generated image. Additionally, markers can be located at the proximal terminator of the shape sensing fiber and one or more surgical instruments. A spatial tracking means can be used to detect and monitor the locations of the markers and determine the position of the instruments relative to the shape sensing fiber proximal terminator and its distal end. The processing means can combine this position data with the reconstructed 3D data to determine the 3D shape generated and shown to a user in an augmented reality display during the procedure using the display.

### BRIEF DESCRIPTION OF THE DRAWINGS

The features and advantages of this disclosure, and the manner of attaining them, will be more apparent and better understood by reference to the following descriptions of the disclosed system and process, taken in conjunction with the accompanying drawings, wherein:

FIG. 1A is a diagram of an exemplary embodiment of a 3D fiber shape sensing and augmented reality system for precise surgery in accordance with at least one embodiment of the present invention.

FIG. 1B is a system diagram of an exemplary embodiment of a 3D fiber shape sensing and augmented reality system of the present disclosure for precise.

FIG. 2A are illustrations pre-operative images of the inserted 3D shape sensing fiber with tissue taken from multiple views by an imaging apparatus for surgical planning to excise a target tissue.

FIG. 2B illustrates an exemplary 3D profile of the target tissue and a generated margin profile using the system of the present disclosure.

FIG. 2C is an illustration of a 3D shape sensing shape fiber of the present disclosure with the registered target tissue and margin are rendered that can be superimposed a display.

FIG. 2D is an illustration of the system of the present disclosure measuring and calculating the real-time distance of the surgical apparatus tip to the generated margin profile.

FIG. 3 shows a flow-chart for a method according to the present disclosure for using a shape sensing fiber for guidance of a surgical instrument during an operation.

FIG. 4 is a block/flow diagram showing a shape sensing system of the present disclosure.

### DETAILED DESCRIPTION OF THE INVENTION

Before any embodiments of the invention are explained in detail, it is to be understood that the invention is not limited in its application to the details of construction and the arrangement of components set forth in the following description or illustrated in the following drawings. The following description includes discussion of figures having illustrations given by way of example of implementations of embodiments of the invention. The drawings should be understood by way of example, and not by way of limitation. As used herein, references to one or more “embodiments” are to be understood as describing a particular feature, structure, or characteristic included in at least one implementation of the invention. Thus, phrases such as “in one embodiment” or “in an alternate embodiment” appearing herein describe various embodiments and implementations of the invention, and do not necessarily all refer to the same embodiment. However, they are also not necessarily mutually exclusive.

The functions of the various elements shown in the FIGS. can be provided through the use of dedicated hardware as well as hardware capable of executing software in association with appropriate software. When provided by a processor, the functions can be provided by a single dedicated processor, by a single shared processor, or by a plurality of individual processors, some of which can be shared. Moreover, explicit use of the term “processor” or “controller” should not be construed to refer exclusively to hardware capable of executing software, and can implicitly include, without limitation, digital signal processor (“DSP”) hardware, read-only memory (“ROM”) for storing software, random access memory (“RAM”), non-volatile storage, etc.

Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure). Thus, for example, it will be appreciated by those skilled in the art that the block diagrams presented herein represent conceptual views of illustrative system components and/or circuitry embodying the principles of the invention. Similarly, it will be appreciated that any flow charts, flow diagrams and the like represent various processes which may be substantially represented in computer readable storage media and so executed by a computer or processor, whether or not such computer or processor is explicitly shown.

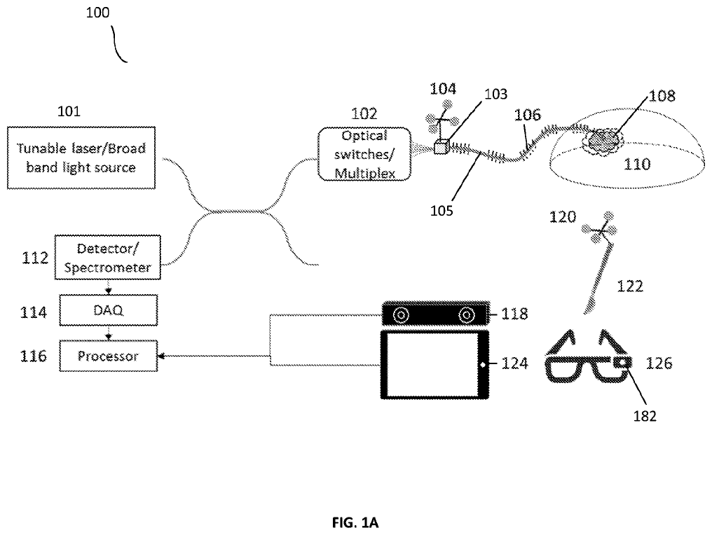

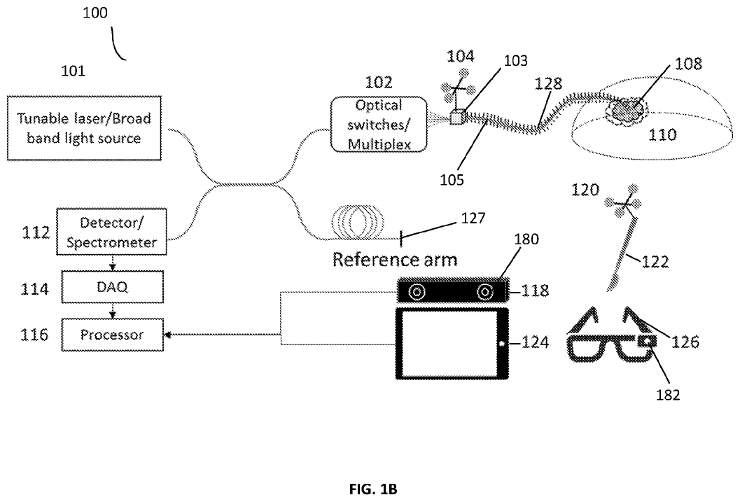

As shown in FIG. 1, a 3D shape sensing fiber and augmented reality system 100 for surgery. The system can include a light source 101, such as a tunable laser/broadband light source, optical switches/multiplex 102, a first group of tracking markers 104, a 3D shape sensing fiber 105 having a Bragg grating 106 (in FIG. 1a) or 128 (in FIG. 1b), a detector element 112, such as a light or EM detector, photodiode, spectrometer, or any of the previously mentioned apparatuses in combination, a data acquisition system (DAQ) 114, a processor 116, a combination of spatial tracking means 118 and a display device, such as a tablet 124 or a head-mounted display (HMD) 126. A second group of tracking markers 120 can be mounted on surgical apparatus 122 and produce a signal. The tracking markers 104 can use any suitable means. In one exemplary embodiment, the tracking markers can actively emit IR or light or passive markers that reflect IR light from an IR light source. Similarly, in some exemplary embodiments, the tracking markers can be EM tracking markers, which can be used as alternatives of the IR markers or in combination with IR markers. The EM markers can provide spatial position information when used with an EM tracking console. Other spatial tracking systems and markers can similarly be used, such as RFID, QR codes, and other suitable tracking means. Similarly, multiple tracking systems can be used in parallel to simultaneously acquire signal from multiple fiber cores or fibers inside the shape sensing fiber for parallel detection. These systems can be used to sense the spatial locations of the markers on the proximal terminator of the shape sensing fiber.

The DAQ 114 can be used to collect data obtained through the detector systems in the regions of interest of a patient. The detector element 112 can measure the real-time reflectivity data from the shape sensing fibers and interfaces with the DAQ 114. The DAQ 114 can digitize the electronic read out signals from the detector element 112 and transmit the digitized signals to the processor 116. The acquired reflectivity data can then be further analyzed and processed by the processor 116 of the system. The processor can be a conventional programmable microprocessor, digital signal processor, microcontroller, or other suitable processing device. The processor can include a memory 200 communicatively coupled to the processor. In some embodiment, the processor 116 can be used to compute the position and orientation of the first group of tracking markers 104 in relation to the second group of markers 120. Similarly, the processor can be used to parse the tracking data collected by the detector, initiate stored modules or algorithms to analyze spatial data into a single coordinate system, and calculate and render images from the data.

One exemplary embodiment of the system of the present disclosure can be used as a surgery guidance system for precise removal of a target tissue. The system can incorporate 3D shape sensing, optical tracking, augmented reality and pre-surgery planning. The 3D shape sensing fiber 105 may be inserted into a target tissue 108 to be removed inside any organ/tissue environment 110. Multiple wavelength light can be coupled into the 3D shape sensing fiber from the light source 101 with either wavelength sweeping using a tunable laser or a broadband light source 101. The 3D shape sensing fiber 105 can include one or more individual sensing fiber cores or fibers, which have fiber Bragg gratings distributed inside. When strain is applied on the fiber 105, the periodicities of the fiber Bragg grating inside can change. As is known, such periodicities changes then modify the reflectivity at different wavelength. The shape sensing fiber can be used in various applications and couple to various elements depending on the desired localization and application. In some exemplary embodiments, the shape sensing fiber 105 can be attached to instrumentation, such as but not limited to, an endoscope, capsule, camera, or tethered to other medical devices or implants. The instrumentation can act as a tracking target when a shape sensing fiber is coupled to the instrumentation.

By measuring the reflectivity changes over wavelength with a detector/spectrometer 112, the strains at different locations on the fiber may be obtained and can be used to reconstruct the 3D shape of the 3D shape sensing fiber 105 from its proximal terminator 103. The optical switches/multiplex 102 can enable either sequentially switching between or multiplex of individual fiber core/fiber inside the shape sensing fiber 105 for signal detection by the detector/spectrometer 112. A data acquisition module 114 can digitize the detected signals and transfers them to the processor 116 for 3D shape reconstruction by the processor. At the same time, one or more markers 104 fixed on the proximal terminator of the 3D shape sensing fiber can produce a signal using any suitable means, such as reflecting a signal from a light source, such as light from IR light source, accompanied with the spatial tracking means 118. A first marker group 104 can be detected and tracked by the spatial tracking means 118 with predefined spatial feature configuration. Pose and position data of the proximal terminator 103 of the 3D shape sensing fiber 105 relative to the spatial tracking means 118 can then be obtained. Combined with the reconstructed 3D shape data of the 3D shape sensing fiber 105 with respect to its proximal terminator, the 3D shape of the shape sensing fiber 105 relative to the spatial tracking means 118 can be obtained.

Additionally, a camera 182 on a tablet 124, a head-mounted display 126, or other imaging device can capture a real-time visual data of the organ/tissue 108 under operation. The 3D shape of the shape sensing fiber 105 can be rendered and superimposed on the visual data and displayed on the display device according to the 3D shape of the shape sensing fiber 105, which can then be viewed and used by the user to carry out the procedure or examination. Both the view of the organ/tissue 108 under operation and the visualization of the 3D shape sensing fiber 105 may be displayed real-time on the screen of the tablet 124, head-mounted display 126, or other display device. A second group of one or more IR markers 120 can be coupled to surgical equipment 122 to form a rigid body to be tracked by a spatial tracking means 118, such as a stereo camera or EM tracking console. The spatial relation of the surgical equipment 122 relative to the 3D shape sensing fiber 105 inserted in the organ/tissue 108 can then be obtained. In various embodiments, the stereo camera can be used for embodiments use IR tracking markers, while an EM tracking console can be used for EM tracking markers.

FIG. 1A and FIG. 1B illustrate exemplary embodiments of the of the present disclosure having an exemplary 3D fiber shape sensing and augmented reality system for surgery with two types of 3D shape sensing fiber: 1) discrete distributed fiber Bragg grating 106 (FIG. 1A, and 2) continuous distributed fiber Bragg grating 128 inside the fiber (FIG. 1B). These two 3D shape sensing fibers achieve the same technique purpose of reconstructing the intraoperative 3D shape of the sensing fiber, while they differ slightly in technology aspect. The system shown in FIG. 1B may further comprise a reference arm 127 to create an interferometric signal by interfering the light reflected from the fiber Bragg gratings and the light from the reference arm 127.

The shape sensing fiber with discrete distributed fiber Bragg grating 106 can have multiple fiber Bragg gratings at different locations, each of which has different periodicity and therefore different center wavelength of reflection. By measuring the reflectivity at different wavelength, the strains on fiber Bragg grating at different locations are obtained, and are used to reconstruct the 3D shape of the fiber.

The shape sensing fiber 105 with continuous distributed fiber Bragg grating 128 has continuous distributed fiber Bragg grating with uniform periodicity. By sweeping the wavelength of the light into the shape sensing fiber, the interferometric signal of the reflected light from FBGs and a reference arm can be recorded by the system. A Fourier transformation can be applied on the interferometric signal, the reflectivity at different wavelength by FBGs can then be retrieved, which can provide the strain information on the fiber at different locations. The collected data can provide more monitoring points of strain on the fiber and more accurate reconstruction of the 3D shape of the sensing fiber 128 and the fibers location within a patient.

The processor 116 can perform various functions and it is contemplated that more than one processor 116 can be employed within the system. Some of the functions performed by the processor 116 include but are not limited to receiving data, storing reference data 212, signal synchronization, initiating programs or modules, such as 3D shape reconstruction 204, optical tracking algorithm 202, spatial geometry calculation module 206, data streaming module 208 and command control module 201 of the system, shown in FIG. 4. Each one of the aforementioned functions can exist stored as a specific module on the memory communicatively coupled to the processor. The optical tracking algorithm 202 collects the optical images of the two groups of markers 104 and 120, and can then calculate the spatial pose and position of them with respect to the spatial tracking means 118. This processed data can then be transmitted to the memory 200. The spatial geometry calculation 206 can transform the 3D shape of the shape sensing fiber 105 from the coordinate system based on proximal terminator 103 to the coordinate system based on the spatial tracking means 118. The data streaming module 208 can transmit the spatial tracking results from the spatial tracking means 118 to the processor 116, the 3D shape of the shape sensing fiber 105 from processor 116 to the display device 124 or 126. The command control module 201 can be configured to collect control commands input by operator and execute through other modules accordingly. Data sources for the processor 116 includes but is not limited to the strain signals on the 3D shape sensing fiber 105 using discrete distributed fiber Bragg grating 106 or continuous distributed fiber Bragg grating 128, optical tracking data from the spatial tracking means 118 and hardware and software information from the tablet 124. The 3D shape reconstruction module, tracking modules and other algorithms are executable code stored in the memory 200 of the processor 116 and various algorithms of each function can be employed in the present invention. The data transfer and streaming media to and from the processor 116 can include but are not limited to Peripheral Component Interconnect Express (PCIe), universal serial bus (USB) wire and local area network (LAN).

The processor/processing means 116 can be more than one computing devices, or a single computing device with more than one microprocessor. The processor 116 can be a stand-alone computing system with internal or external memory, a microprocessor and additional standard computing features. The processor 116 can be selected from the group comprising a PC, laptop computer, microprocessor, or alternative computing apparatus or system.

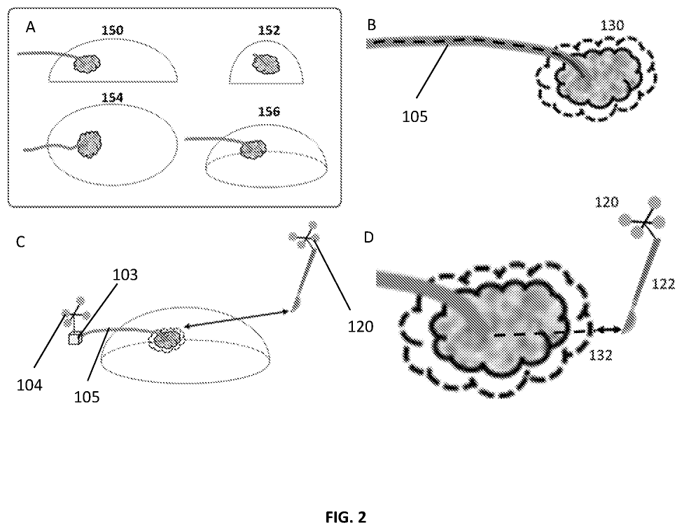

FIG. 2 shows a system diagram of using preoperative images and 3D shape sensing fiber to perform surgical planning, and to excise the target tissue out in accordance with at least one embodiment of the present invention. FIG. 2A illustrates pre-operative images 150, 152, 154 of the inserted 3D shape sensing fiber with tissue taken from multiple views by an imaging apparatus, which can include but is not limited to ultrasound, mammogram, X-ray, and magnetic resonance imaging (MRI). During the surgical planning phase, operators/users can identify the target tissue to remove and mark the contours on the pre-operative images. With the contours of the target tissues on the registered images, a 3D shape/profile of the tumor 156 can be reconstructed and approximate the tumor's 3D location using a developed algorithm of the reconstruction module. Further, with a margin defined by operators, a 3D profile with margin 130 can be generated by the system for complete removal during an operative procedure. The margin can be manually defined or automatically generated by the processor of the system. Additionally, the memory can store pre-determined margin ranges based upon various types of procedures and optimal excision margins. The margin 130 can be superimposed over the image to provide a user a visual guide as to where to excise the tissue.

FIG. 2B shows an exemplary 3D profile of the target tissue and the generated margin profile (dashed contours). The thickness of the margin is tunable according to the operators' need. Also, the 3D profile of the part of the 3D shape sensing fiber inside the generated target tissue profile is generated and stored (solid lines in FIG. 2B). During the surgery, the intraoperative 3D profile of the whole 3D shape sensing fiber 105 or 128 is reconstructed by methods described above. With the assistance of a developed registration algorithm, the 3D profile of the target tissue and the margin is registered on the reconstructed 3D shape of the shape sensing fiber 105 through the fitting of the pre-operative 3D profile to the intraoperative one of the shape sensing fiber 105 having a Bragg grating 106 or 128. FIG. 2C shows the intraoperative reconstruction of the 3D shape sensing shape fiber 105 having a Bragg grating 106 or 128 with the registered target tissue and margin are rendered and superimposed on the view of the tablet 124 or the head-mounted display 126. The visualization guidance of the target tissue with the margin could guide operators to perform fast and precise removal of the target tissue. Additionally, the spatial tracking means tracks the surgical apparatus 122 through the second group of trackers 120 on the surgical apparatus 112. As shown in FIG. 2D, the real-time distance of the surgical equipment tip 132 to the generated margin can be calculated. Feedback provided to the operators includes but is not limited to visual and audio, once the surgical equipment tip goes into the margin area, i.e. the real-time distance 132 is less or equal to zero. Similarly, the display image can be superimposed over the real-time view of the tissue being operated or examined in real-time. In one exemplary embodiment, preoperative images of target tissue can be registered or displayed through augmented display means for surgical guidance. Information, such as shape and geometry of targets to be tracked can be registered, rendered and superimposed on the view of the tablet 124 or the head-mounted display 126. The targets to be tracked can include but are not limit to tissue to be removed, endoscopic devices, medical devices or implants.

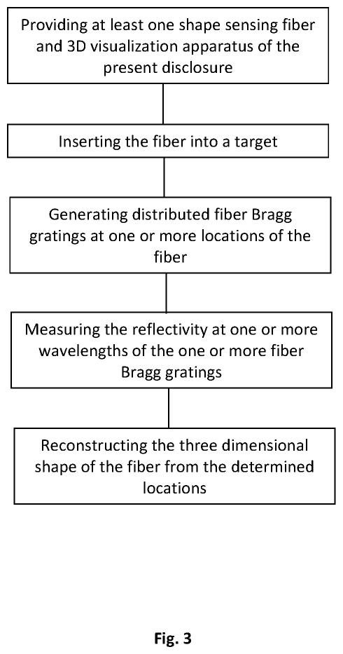

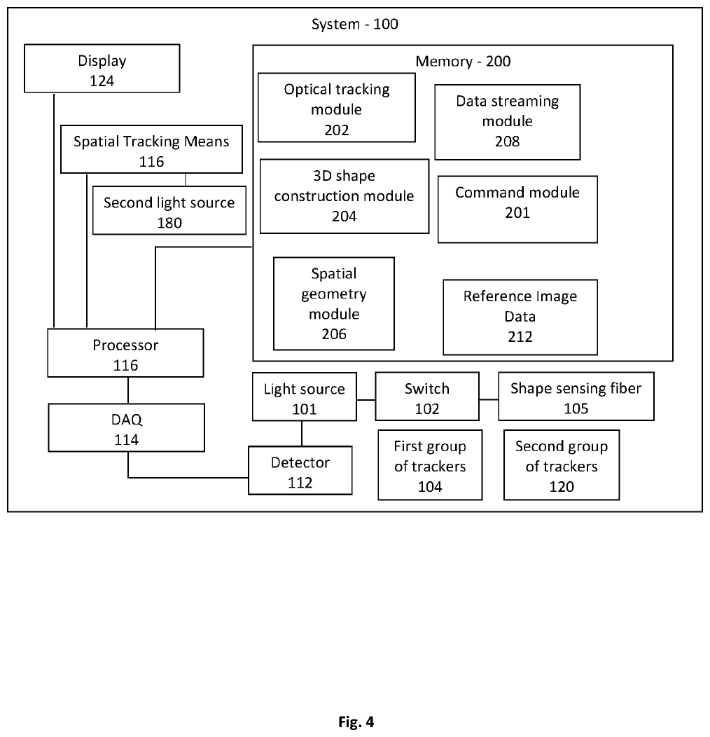

FIG. 3 shows an exemplary method of the system of the present disclosure. A method for providing an augmented reality surgical system can include first providing at least one shape sensing fiber and 3D visualization system as disclosed herein. A shape sensing fiber having Bragg grating can then be inserted into a target tissue or pre-determined area of a patient. In addition, the shape sensing fiber having Bragg grating can be mounted or inserted in a target surgical instrument to be tracked. A light source can be used and ran through the shape sensing fiber, wherein the fiber has fiber Bragg gratings at one or more locations of the fiber within the patient. The reflectivity at one or more wavelengths of the one or more fiber Bragg gratings at one or more locations of the fiber with the patient can then be measured. The strains on the fiber Bragg gratings at different locations can be determined. A three dimensional shape of the fiber from the determined locations can then be generated. A display can be used to present the generated image. Additionally, markers can be located at the proximal terminator of the shape sensing fiber and one or more surgical instruments. A spatial tracking means can be used to detect and monitor the locations of the markers and determine the position of the instruments relative to the shape sensing fiber proximal terminator and its distal end. The processing means can combine this position data with the reconstructed 3D data to determine the 3D shape generated and shown to a user in an augmented reality display during the procedure using the display. The method can further include sweeping the wavelength of the light source into the shape sensing fiber and measuring the Fourier transformation of the reflectivity at one or more wavelengths to determine the strain on the fiber at different locations.

In some exemplary embodiments, the pose and position of the proximal terminator within the target can be achieved using by initiating the reconstruction module. Pose and position data and reconstructed three dimensional shape data can be combined and analyzed by the processor to determine the three dimensional shape of the sensing fiber relative to the spatial tracking means. The location of the second group of markers on a surgical apparatus can be tracked using the spatial tracking means. The location data can be used to determine the spatial relationship between a surgical apparatus relative to the shape sensing fiber in the target. A visual camera can be used capture real-time image data of the tissue being examined by the user. The processor can then use the real-time image data collected by the camera and superimpose the rendered three dimensional visualization data generated over the real-time image data generated by the camera. The processor can the display the superimposed data on a display device, such as a tablet or heads-up display device. Additionally, the trackers on surgical apparatus can be tracked in relation to the shape sensing fiber and tracking target. In the case of the tracking target being tissue desired to be removed, the system can alert the user of the surgical equipment when the users is proximate to the desired margin. If the user begins to excise within the margin, the system can trigger an alert to the display or other alerting means that the user has is within the desired margin and not outside the desired margin. Similarly, if a user is too far from the desired margin, the display can provide visual or audio feedback to the user.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

What is claimed is:

1. A surgical shape sensing apparatus comprising:
a light source;
a shape sensing fiber having a proximal terminator;
at least one optical switch;
a first group of tracking markers;
a second group of tracking markers;
a detector element;
a data acquisition system;
a processor;
a memory communicatively coupled to the processor, wherein said memory store one or more modules;
a spatial tracking means configured to detect and track the first and second group of one or more markers within a predetermined area; and
a display.

2. The apparatus of claim 1, wherein said light source is a broadband and tunable light source, wherein said first group of detector elements are fixed on the proximal terminator of the shape sensing fiber, and wherein the second group of tracking markers are coupled to surgical apparatus.

3. The apparatus of claim 2, wherein the switch configured to sequentially switch between a multiplex of individual fibers inside the shape sensing fiber for signal detection.

4. The apparatus of claim 3, wherein said shape sensing fiber comprises a plurality of individual sensing fiber cores having a fiber Bragg grating distributed within said fiber.

5. The apparatus of claim 4, wherein said data acquisition module configured to digitize the detected signals and communicate the digitized signals to the processing means, wherein the processing means is configured to reconstruct a 3D shape based on the signals

6. The apparatus of claim 4, wherein the first shape sensing fiber is integrated into a target, wherein the target consists of one or more of the following:
a pre-determined tissue, endoscopic devices, endoscopic capsules, medical devices or a medical implants.

7. The apparatus of claim 6, wherein the first group of tracking markers and second group of tracking markers are infrared markers, wherein said markers are configured to actively emit light at a pre-determined wavelength range.

8. The apparatus of claim 6, wherein the first group of tracking markers and second group of tracking markers are infrared markers configured to passively reflect light from generated by the light source at a pre-determined wavelength.

9. The apparatus of claim 6, wherein the first group of tracking markers and second group of markers are electromagnetic markers.

10. The apparatus of claim 7, wherein the spatial tracking means is a stereo camera configured to detect and track the first group of markers, wherein said data acquisition module is configured to digitize the detected signals and communicate the digitized tracking data to the processing means, wherein the processing means is configured to create reconstructed 3D visual data of the shape sensing fiber using digitized tracking data, wherein the stereo camera is further configured to track the second group of markers on the surgical apparatus, wherein the obtained tracking data is communicated to the processing means and the spatial relation of the surgical apparatus relative to the 3D shape sensing fiber inserted in the tissue is obtained.

11. The apparatus of claim 9, where in the spatial tracking means is an electromagnetic tracking module that is configured to detect and track one or more of the markers, wherein said data acquisition module is configured to digitize the detected signals and communicate the digitized signals to the processing means, wherein the processing means is configured to create reconstructed 3D data using digitized tracking data.

12. The apparatus of claim 10, further comprising a camera configured to obtain real-time visual data of the tissue being examined.

13. The apparatus of claim 11, wherein the display is configured to render the shape of the sensing fiber and superimpose the shape over the real-time visual data of the tissue being operated or examined in real-time.

14. A shape sensing apparatus for tissue and surgical procedures comprising:
a processing means;
a light source;
a tracking target comprising least one shape sensing fiber, wherein said shape sensing fiber comprises a plurality of individual sensing fiber cores having a fiber Bragg grating distributed within the fiber;
an optical switch configured to sequentially switch between a multiplex of individual fibers inside the shape sensing fiber for signal detection;
a detector configured to detect the fiber signals produced by the shape sensing fiber;
a data acquisition module configured to digitize the detected signals and communicate the digitized signals to the processing means, wherein the processing means is configured create reconstructed 3D data using digitized detected;
a first group of one or more markers, wherein said first group of one or more markers are fixed on a proximal terminator of the shape sensing fiber and configured to produce a signal;
a spatial tracking means configured to detect and track the first group of one or more markers within a predetermined area;
wherein the processing means is configured to determine a set of position data using the pose and position of the proximal terminator relative to the spatial tracking means, and combine the position data with the reconstructed 3D data to determine the 3D shape of the sensing fiber relative to the spatial tracking means;
a camera configured to obtain real-time visual data of the tissue being examined;
a display configured to render the shape and geometry of the tracking target and superimpose the shape over the real-time view of the tissue being examined in real-time; and
a second group of one or more markers mounted to a surgical instrument, wherein the second group of one or more markers is tracked by the spatial tracking means, wherein the obtained tracking data is communicated to the processing means and the spatial relation of the surgical equipment relative to the 3D shape sensing fiber inserted in the tissue is obtained.

15. The apparatus of claim 10, wherein the tracking target is coupled to at least one or more of the following:
a pre-determined tissue area, endoscopic devices, endoscopic capsules, medical devices or a medical implant.

16. The apparatus of claim 14, wherein said processing means further comprises a memory having a shape construction module to reconstruct three dimensional shapes resulting in 3D shape data of the fiber from determined locations; a spatial geometry module configured to determine the location of the shape sensing fiber relative to the spatial tracking means; an optical tracking module configured to collect the optical images of the first group of markers and the second group of markers to calculate the spatial pose and position data of the markers with respect to the spatial tracking means; and a data streaming module configured to transmit the spatial pose and position data, the 3D shape data to be displayed on the display.

17. A method of providing an augmented reality surgical system comprising:
providing at least one shape sensing fiber and 3D visualization system as disclosed;
integrating the fiber into a target;
generating distributed fiber Bragg gratings at one or more locations of the fiber;
measuring the reflectivity at one or more wavelengths of the one or more fiber Bragg gratings;
determining the strains on fiber Bragg gratings at different locations; and
reconstructing the three dimensional shape of the fiber from the determined locations;

18. The method of claim 17, further comprising sweeping the wavelength of light into the shape sensing fiber and measuring the Fourier transformation of the reflectivity at the one or more wavelengths to determine the strain on the fiber at different locations.

19. The method of claim 17, further comprising
providing a spatial tracking means;
providing a first group of one or more markers coupled to a proximal terminator of the fiber;
producing a signal of first group of one or more markers;
detecting and tracking the signal of the first group of one or more markers with the spatial tracking means;
determining the pose and position of the proximal terminator within the target using the reconstruction module;
combining the pose and position data and the reconstructed three dimensional shape data;
determining the three dimensional shape of the sensing fiber relative to the spatial tracking means;
providing a second group of one or more infrared markers coupled to one or more surgical apparatuses;
tracking the location of the second group of one or more markers using the spatial tracking means; and
determining the spatial relationship between the surgical apparatuses relative to the shape sensing fiber in the tracking target.

20. The method of claim 19, further comprising:
providing a camera for capturing real-time image data of the tissue being examined;
superimposing the rendered three dimensional visualization data over the real-time image data; and
displaying the superimposed data on a display device.
